(19) United States
(12) Patent Application Publication
(10) Pub. No.: US 2025/0296907 A1
(43) Pub. Date: Sep. 25, 2025

(54) METHOD FOR OBTAINING ACETOIN

(71) Applicant: Lesaffre et Compagnie, Paris (FR)

(72) Inventors: Pierre TIBAYRENC, Arcangues (FR); Ahmed YAHIA

(21) Appl. No.: 18/857,094

(22) PCT Filed: Apr. 13, 2023
(86) PCT No.: PCT/FR2023/050528

§ 371 Date: Oct. 15, 2024

(30) Foreign Application Priority Data
Apr. 15, 2022 (FR) FR2203539

(51) Int. Cl. C07C 45/78; C07C 49/17

(52) U.S. Cl. CPC C07C 45/786; C07C 49/17

(57) ABSTRACT

The invention relates to a method for obtaining acetoin from a medium containing same, the method comprising a step of dehydration by pervaporation using a hydrophilic membrane. With this method, the acetoin can be satisfactorily isolated and purified, both in terms of quality and yield. Furthermore, with the method of the invention it is possible to dispense with the use of solvents and prevent impurities from being generated.

### FIELD OF THE INVENTION

The present invention relates to a method for obtaining acetoin from a medium comprising same, the method comprising a dehydration step by pervaporation using a hydrophilic membrane. Such method serves to isolate and purify acetoin satisfactorily, both in terms of quality and yield. Furthermore, the method eliminates the use of solvents and prevents the generation of impurities.

### TECHNOLOGICAL BACKGROUND

Acetoin (3-hydroxybutanone) is a chiral organic molecule of formula C₄H₈O₂ belonging to the class of hydroxylated ketones and comprising two enantiomeric forms. Acetoin has two functional groups, a hydroxyl group and a carbonyl group, which make acetoin particularly reactive and prone to decomposing, in particular into acetaldehyde.

Acetoin is found in trace amounts in many foods, in particular grapes, raspberries, apples, bananas, corn, honey and coffee. The molecule is also present in certain cheeses or certain meats. Finally, the molecule is also a synthetic intermediate in the metabolism of sugars of certain microorganisms.

Acetoin is, along with diacetyl (2.3-butanedione), one of the molecules that imparts butter the characteristic flavor thereof. Acetoin can be used as a flavoring agent.

Acetoin is frequently used in particular in the food industry, more particularly in the fields of dairy products, beverages, bread and bakery, and pastry, to impart a “butter flavor” to products containing same. In the above field, the regulations require the use of acetoin with a high degree of purity, e.g. at least 95% (as per Regulation (EC) No 1334/2008).

Acetoin is also used in other fields, more particularly in the fields of perfumery, pharmacy and of the use of tobacco.

Acetoin can be obtained by means of chemical synthesis methods or biological methods.

Chemical synthesis methods can use, e.g., 2,3-butanedione and 2,3-butanediol as raw materials.

The biological methods may use wild microorganisms, selected microorganisms or recombinant microorganisms.

Recombinant microorganisms are known, which do not naturally produce acetoin or produce acetoin in too low a yield and/or as a synthesis intermediate. For example, recombinant microorganisms have been obtained from the following microorganisms: Bacillus subtilis, Bacillus pumilus, Bacillus methanolicus, Bacillus licheniformis, Corynebacterium glutamicum, Escherichia coli, Lactococcus lactis, Saccharomyces cerevisiae and Serratia marcescens.

Irrespective of the production method, the quantity and quality of the product obtained may not be sufficient, in particular for use in the fields of food-processing and pharmacy. Furthermore, a medium containing impurities, in particular acetoin by-products, organic residues (biomass) can be obtained when obtained with a biological method, as well as other molecules. It is thus necessary to isolate and purify acetoin from the medium containing same.

A number of purification techniques are known, e.g. distillation, liquid-liquid extraction or salting-out extraction.

The effectiveness of the techniques, more particularly distillation, may be limited by the fact that acetoin forms an azeotropic mixture with water, the separation being thereby hindered or limited when the liquid phase and the gas phase reach the same water/acetoin composition.

The use of solvents is not recommended, in particular when acetoin is intended for the food-processing industry. Indeed, for regulatory reasons, only certain solvents can be used for a product containing acetoin to be labeled as “natural”. Among the available solvents, only ethyl acetate is both compliant with the European regulations and particularly suitable for the purification of acetoin. However, the use of such solvent requires large quantities, making the method less economically viable, on an industrial scale, for efficient extraction. Furthermore, such solvent does not allow the use of the American NOP (National Organic Program) label.

The international application WO 2021/060334 A1 published on Apr. 1, 2021 discloses a purification method using a tangential filtration technology implemented by a nanofiltration step, followed by a reverse osmosis step. However, the method does not efficiently achieve residual moisture contents of less than 50%.

Thereby, there is a real need to provide a method for obtaining acetoin that can be used directly in processed food preparations. There is also a real need to provide a method for obtaining acetoin at a high yield and at an affordable cost. There is also a real need to provide a method implemented without using solvents, in particular ethyl acetate. There is also a real need to provide a method for obtaining acetoin that can be labeled as “natural”.

### SUMMARY OF THE INVENTION

The invention relates firstly to a method for obtaining acetoin from a medium to be treated, the method comprising a dehydration step by pervaporation using a hydrophilic membrane, in order to obtain a dehydrated medium comprising at least 70% acetoin, by total weight of the treated medium.

In embodiments, the hydrophilic membrane is chosen from the group consisting of hydrophilic polymeric membranes, hydrophilic inorganic membranes, hydrophilic two-dimensional material membranes, hydrophilic mixed matrix membranes, or membranes derived therefrom.

In embodiments, the hydrophilic membrane is a hydrophilic silica membrane or a hydrophilic membrane derived therefrom.

In embodiments, the hydrophilic silica membrane is obtained from organoalkoxysilane precursors.

In embodiments, an underpressure of −700×10⁵ mPa or more is applied to the permeation surface of the hydrophilic membrane.

In embodiments, the dehydration step is carried out in the absence of solvents and/or inorganic salts.

In embodiments, the method comprises a step of pretreatment of the medium to be treated, which is carried out, before the dehydration step, using a technique chosen from the group consisting of centrifugation, frontal filtration, microfiltration and nanofiltration.

In embodiments, the method comprises a post-treatment step of the treated medium, which is carried out, after the dehydration step, by distillation.

In embodiments, the medium comprising acetoin is obtained by a biological method carried out using recombinant microorganisms.

In embodiments, the recombinant microorganisms are yeasts belonging to the genus Saccharomyces.

Secondly, the invention relates to acetoin, obtained from the method described thereafter.

Thirdly, the invention relates to the use of a hydrophilic membrane, as described thereafter, to obtain acetoin.

The present invention makes it possible to meet the needs expressed hereinabove.

Surprisingly, the inventors have demonstrated that acetoin can be satisfactorily isolated and purified, both from the point of view of quality and yield, by selecting hydrophilic membranes. When using such membranes, only water and hydrophilic compounds migrate through the membrane into the permeate (filtrate), whereas acetoin remains in the retentate (treated medium), which leads to progressive and satisfactory dehydration of acetoin. Such pervaporation dehydration technique also makes it possible to dispense with the separation constraints associated with the formation of azeotropic mixtures.

The liquid-liquid extraction technique, commonly used, has the disadvantage of being carried out with solvents, e.g. ethyl acetate, used in large quantities. Furthermore, disadvantageously, the inventors have demonstrated that such technique leads to the formation of impurities, e.g. methylallyl acetate, a reaction product between acetoin and ethyl acetate. Finally, such technique does not make it possible to obtain acetoin that can be labeled as “natural”.

The technique according to the application PCT WO 2021/060334 A1 makes it possible to dispense with the use of solvents, but is not economically viable, in that the acetoin yield is too low, because of the formation of an azeotropic water-acetoin mixture and the removal of a significant part of the acetoin with the water.

The use of the pervaporation dehydration technique according to the invention, using a hydrophilic membrane, makes it possible to dispense with the use of solvent, to prevent the generation of impurities in the final product, to circumvent the separation difficulties linked to the formation of an azeotropic mixture and to obtain a satisfactory yield of acetoin.

Furthermore and counter-intuitively, the inventors have also demonstrated that the use of a hydrophilic membrane makes it possible to obtain a method with improved performance compared to a method using an organophilic membrane, i.e. a higher yield and a higher degree of dehydration, although water is initially predominant in the medium.

### BRIEF DESCRIPTION OF FIGURES

FIG. 1 shows a graph of the concentration of acetoin and water (mass percent) in the retentate as a function of time (minute).

FIG. 2 shows a graph of the acetoin concentration in the retentate (mass percent) and the underpressure at the permeation surface (x105 mPa) as a function of time (minute).

### DETAILED DESCRIPTION

The invention is now described in greater detail and in a non-limiting manner in the following description.

In the description, unless otherwise stated, all the percentages indicated are mass percentages.

In a first aspect, the present invention relates to a method for obtaining acetoin from a medium comprising same (medium to be treated), the method comprising a dehydration step by pervaporation using a hydrophilic membrane.

### Dehydration Step

The dehydration step consists in bringing the medium to be treated into contact with a first surface of the hydrophilic membrane and in applying an under-pressure to the permeation surface (second surface) of the hydrophilic membrane.

The under-pressure applied at the permeation surface of the hydrophilic membrane is −700×10⁵ mPa or more, preferentially from −300×10⁵ to −0.1×10⁵ mPa, very preferentially from −100×10⁵ to −10⁵ mPa.

The under-pressure is defined in relation to the pressure at the surface in contact with the medium to be dehydrated.

The temperature applied to the feed is 150° C. or less, preferentially 5 to 100° C., very preferentially 30 to 90° C.

Thus, a permeate is gradually formed comprising water and hydrophilic compounds which have migrated through the hydrophilic membrane (medium to be removed), whereas the residual medium, comprising acetoin, forms the retentate (treated medium).

### Hydrophilic Membrane

“Hydrophilic membrane” refers to a membrane which preferentially lets through the permeate, water and hydrophilic molecules.

The hydrophilic membrane may be chosen from the group consisting of hydrophilic polymer membranes, hydrophilic inorganic membranes, membranes based on hydrophilic two-dimensional materials (two-dimensional membranes), hydrophilic mixed matrix membranes or hydrophilic membranes derived therefrom; preferentially, the hydrophilic membrane being an inorganic membrane or a hydrophilic membrane derived therefrom.

The hydrophilic inorganic membrane may be chosen from the group consisting of hydrophilic zeolite membranes, hydrophilic silica membranes, hydrophilic metal-organic framework (MOF) membranes, hydrophilic covalent organic framework (COF) membranes or hydrophilic membranes derived therefrom; preferentially, the hydrophilic inorganic membrane is a membrane based on hydrophilic silica or a hydrophilic membrane derived therefrom; very preferentially, the hydrophilic inorganic membrane is a hydride membrane based on hydrophilic silica. Silica hydride membranes are referred to indiscriminately as organic-inorganic silica membranes, organo-silica membranes or organosilica hydride membranes.

The hydrophilic silica hydride membrane may be chosen from the group consisting of silica hydride membranes with hydrophilic pendant groups (“pendant-type organosilica membranes”) or hydrophilic bridged silica hydride membranes (“bridged-type organosilica membranes”); preferably hydrophilic bridged silica-based hydride membranes.

Silica hydride membranes are known (cf. the review by X. Ren with the title “Organosilica-based membranes in gas and liquid-phase separation”, Membranes, 2019, 9, 107; the book entitled “Pervaporation, vapor permeation and membrane distillation—principles and applications”, Ed. Woodhead Publishing Series in Energy, 2015, including chapter 5 entitled “Next-generation pervaporation membranes: recent trends, challenges and perspectives”, 107-141; Article by G. Liu with the title “Pervaporation membrane materials: Recent trends and perspectives”, Journal of Membrane Science, 2021, 636).

Bridged-type organosilica membranes can be obtained from organoalkoxysilane precursors (monomers), i.e. alkoxysilane precursors comprising organic moieties.

The organoalkoxysilane precursor may be chosen from organoalkoxysilane precursors comprising one, two, three or more than three silica atoms.

The organoalkoxysilane precursor comprising a silica atom may be chosen from the group consisting of methyltrimethoxysilane (MTMS), methyltriethoxysilane (MTES), phenyltriethoxysilane (PhTES), hydroxymethyl (triethoxy) silane (HMTES), (3-aminopropyl) triethoxysilane (APTES) and mixtures thereof.

The organoalkoxysilane precursor comprising two silica atoms may be selected from the group consisting of bis (triethoxysilyl) methane (BTESM), 1-2-bis(triethoxysilyl)ethane (BTESE), 1,3-bis(triethoxysilyl)propane (BTESP), 1,8-bis(triethoxysilyl)octane (BTESO), 1-2-Bis(triethoxysilyl)ethylene (BTESEthy), le 1,2-bis(triethoxysilyl)acetylene (BTESA), 1,4-bis(triethoxysilyl)diacetylene (BTESDA), le bis(trimethoxysilyl)norbornane (BTMS-Nor), 1,2-bis(triethoxysilyl)benzene (BTESB), 2,5-bis[2-triethoxysilyl)ethyl]-1,4-dioxane (BTES-ED), bis[3-(triethoxysilyl)propyl]amine (BTESPA), 1,6-diacetoxy-3,4-bis(triethoxysilyl)hexa-2,4-diene (BTES-Ac), 4,6-bis(3-triethoxysilyl-1-propoxy)-1,3-pyrimidine (BTPP), bis(triethoxysilyldimethyl/dipropyl-N,N′-oxalylurated (BTESMOU/BTESMPU) and mixtures thereof; preferentially, the organoalkoxysilane comprising two silica atoms is chosen from the group consisting of BTESM, BTESE a mixture thereof; very preferentially, the organoalkoxysilane comprising two silica atoms, is BTESE.

The organoalkoxysilane precursor may be chosen from the group consisting of TTESPT, TESE-POSS, MTMS, MTES, PhTES, HMTES, APTES, BTESM, BTESE, BTESP, BTESO, BTESEthy, BTESA, BTESDA, BTMS-Nor, BTESB, BTES-ED, BTESPA, BTES-Ac, BTPP, BTESMOU/BTESMPU, TTESPT, TESE-POSS and mixtures thereof; preferentially, the organosilane precursor is chosen from the group consisting of BTESM, BTESE and a mixture thereof; very preferentially, the organoalkoxysilane precursor is BTESE.

The organic moiety can be used to functionalize the alkoxysilane precursors.

The organic group used to form organic bridges between the organoalkoxysilane precursors may be chosen from the group consisting of methane (CH4), ethane (CH3-CH3), propane (CH3-CH2-CH3), hexane (CH3-(CH2)4-CH3), octane (CH3-(CH2)6-CH3), phenyl, ethylene (CH2=CH2), acetylene (CHCH), diethylenedioxane, norbornane, triazine, pyrimidine and mixtures thereof. For example, organic bridges such as —CH2-, —CH2-CH2-, —CH2-CH2-CH2-, —(CH2)₆-, —(CH2)₈, phenyl, —CH═CH—, an acetylene bridge, a diethylenedioxane bridge, a norbornane bridge, a triazine bridge, a pyrimidine bridge and mixtures thereof, are obtained.

Silica-based hydride membranes can be subjected to additional treatments, e.g. with metals.

Hydrophilic membranes are commercially available, e.g. under the name Hybsi@ from Hybsi@ (Pervatech@).

### Medium to be Treated

The medium to be treated corresponds to the starting product (non-pretreated medium) or alternatively to the intermediate product (pretreated medium) which has undergone a pretreatment step beforehand.

The terms “medium”, “composition”, “solution” and “substrate” may be used without distinction to designate the medium to be treated (and, where appropriate, the treated medium).

The medium to be treated may comprise from 2 to 50%, preferentially from 6 to 30%, of acetoin, per total weight of the medium. As an example, the medium to be treated may comprise 1 to 5%, or from 5 to 10%, or from 10 to 15%, or from 15 to 20%, or from 20 to 25%, or from 25 to 30%, or from 30 to 35%, or from 35 to 40%, or from 40 to 45%, or from 45 to 50% of acetoin by total weight of the medium. Acetoin may be present in the form of the (3R)-3-hydroxybutanone enantiomer (natural enantiomer) thereof, the (3S)-3-hydroxybutanone enantiomer thereof or a mixture of the two enantiomers (including racemic mixture).

The medium to be treated is an aqueous medium. The medium to be treated may comprise at least 15%, preferentially at least 45%, very preferentially at least 65%, of water, per total weight of the medium.

The medium to be treated can be obtained from a chemical synthesis method or from a biological method.

### Treated Medium

After carrying out the dehydration step, a treated medium (corresponding to the retentate), is obtained.

The treated medium may comprise at least 70% (from 70 to 99%), preferentially at least 80% (from 80 to 99%), very preferably at least 90% (from 90 to 99%) of acetoin, per total weight of the medium. As an example, the dehydrated medium may comprise from 70 to 75%, or from 75 to 80%, or from 80 to 85%, or from 85 to 90%, or from 90 to 95%, or from 95 to 99%, of acetoin, by total weight of the medium.

As a corollary, the treated medium may comprise 30% or less (from 1 to 30%), preferentially 20% or less (from 1 to 20%), very preferentially 10% or less (from 1 to 20%), of water and additional compounds (impurities), by total weight of the medium.

### Filtration Agents (Absence)

The dehydration step is carried out in the absence of solvents, e.g. ethyl acetate, methyl tert-butyl ether, butyl acetate, dimethyl carbonate, diethyl ether, diethyl carbonate, ethanol, methanol, acetone, isopropanol, acetonitrile, dichloromethane and/or chloroform. More particularly, the dehydration step is carried out in the absence of ethyl acetate.

The dehydration step is carried out in the absence of inorganic salts, e.g. ammonium sulfate, magnesium sulfate, sodium sulfate, copper sulfate, calcium chloride, potassium hydrogen phosphate (dipotassium phosphate), potassium pyrophosphate, sodium carbonate and/or potassium carbonate.

### Pretreatment Step

The method may comprise a pretreatment step of the medium to be treated, carried out before the dehydration step. In such embodiment, the medium to be treated is pretreated and the pretreated medium thereby obtained is subsequently dehydrated.

A pretreatment step can be used to remove suspended solids in the medium to be treated. More particularly, a pretreatment step can be used when acetoin is produced by a biological method and the medium to be treated thus comprises biomass.

When the acetoin-containing medium is obtained from a biological method, same may comprise biomass. “Biomass” refers to the organic matter of bacterial or fungal, plant or animal origin, preferentially organic matter of bacterial or fungal origin. When the medium is obtained from a biological method using bacteria or fungi including yeasts, the biomass may comprise whole microorganisms (living or dead), residues of the microorganisms (residues of nuclei, organelles, cytoplasm, membrane, etc.) and molecules produced by same (proteins, carbohydrates, lipids, etc.). The medium to be treated (not pretreated) may comprise from 1 to 20%, preferentially from 1 to 10%, of dry biomass, per total weight of the medium.

The pretreatment step can make it possible to remove at least 80%, preferentially at least 90%, of the dry biomass contained in the medium to be dehydrated.

The pretreatment step can be chosen by techniques consisting of centrifugation, frontal filtration, microfiltration, ultrafiltration, nanofiltration or any other conventional clarification technique.

### Post-Treatment Step

The method may comprise a post-treatment step of the treated medium (retentate), carried out after the dehydration step.

A post-treatment step can be used to increase the purity of acetoin.

The post-treatment step can be carried out by distillation.

The method according to the invention is particularly advantageous, in that it serves to dispense with the difficulties encountered in the implementation of direct distillation, because of the formation of an azeotropic water-acetoin mixture.

### Method of Chemical Synthesis of Acetoin

The medium containing acetoin can be obtained by a chemical synthesis method.

### Biological Method for Obtaining Acetoin

The medium containing acetoin can be obtained by a biological method.

The biological method can be carried out using microorganisms, in particular wild microorganisms, selected microorganisms or recombinant microorganisms.

Wild microorganisms are microorganisms that naturally produce acetoin at usable concentrations. The selected (non-genetically modified) microorganisms are microorganisms with improved performance in terms of production of acetoin.

Recombinant (genetically modified) microorganisms—not producing acetoin naturally or producing acetoin in too low a yield and/or as a synthesis intermediate—may be chosen from the group consisting of Bacillus subtilis, Bacillus pumilus, Bacillus methanolicus, Bacillus licheniformis, Corynebacterium glutamicum, Escherichia coli, Lactococcus lactis, Serratia marcescens and yeasts of the genus Saccharomyces, preferentially a microorganism being of the genus Saccharomyces, very preferentially Saccharomyces cerevisiae.

### Acetoin Thereby Obtained

The method according to the invention makes it possible to obtain a product comprising at least 70% of acetoin, by total weight of the treated medium.

### EXAMPLES

### Example According to the Invention: Method for Obtaining Acetoin Using a Hydrophilic Membrane

### Materials

Medium to be purified: Aqueous solution comprising 10% acetoin, by total weight of the aqueous solution
Hydrophilic membrane: Hybsi RAR membrane marketed by Hybsi®
Dehydration device: PTU-044 device

### Parameters of the Pervaporation Dehydration Step

Temperature of the medium: 100° C.
Linear velocity (velocity) of feed in the medium:
1-2 m/s
Permeate under-pressure (vacuum pressure): −20.10⁵ mPa

### Parameters of the Distillation Post-Treatment Step

Distillation mode: batch
Column feature: 20 stages
Vacuum applied: 150 mbar
Vapor temperature: Between 44 and 86° C.
Reflux ratio: between 1 and 5

### Water Content Measurement

The water content in the retentate and in the filtrate is measured by the Karl Fischer filtration method (KF titration).

### Measurement of Acetoin Content

The acetoin content is measured by HPLC and/or gas chromatography.

### Dehydration Method

The purpose the method is to obtain the permeation of water from the medium to be treated through the hydrophilic membrane to the permeate and as a corollary the dehydration of acetoin (retentate).

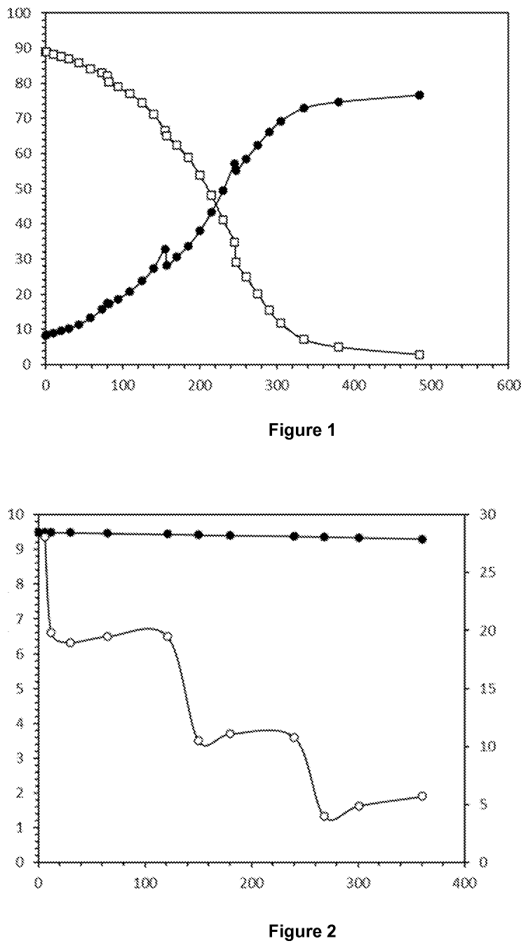

As shown in FIG. 1, which shows a graph of the concentration of acetoin and water (mass percent) in the retentate as a function of time (minutes), the concentration of water (white squares) decreases and, on the other hand, the concentration of acetoin (black circles) increases in the retentate over time. After 485 minutes of treatment, the water concentration decreases to about 1.1%, by total weight of retentate, while the acetoin concentration increases to about 77%, the difference corresponding to organic residues other than acetoin.

The implementation of a post-treatment step by distillation makes it possible to obtain an acetoin having a purity of 98.5% with a yield of 80.5%.

### Comparative Example: Method of Obtaining Acetoin Using an Organophilic Membrane

### Materials

Medium to be purified: Aqueous solution comprising 10% acetoin, by total weight of the aqueous solution
Organophilic membrane: PDMS-FS membrane marketed by Pervatech®
Extraction device: PTU-044 device

### Parameters

Temperature of the medium: 55° C.
Feed rate in the medium: 200 L/h
Permeate vacuum pressure: from 20×10⁵ mPa to 4×10⁵ mPa

### Liquid-Liquid Extraction Method

The purpose of the method is to obtain the permeation of acetoin from the medium comprising same through the organophilic membrane to the filtration agent.

As shown in FIG. 2, which shows a graph relating to acetoin concentration in the retentate (left ordinate, mass percent) and the under-pressure at the permeation surface (right ordinate, X×10⁵ mPa) versus time (minutes), filtration is not sufficiently selective, in that migration of acetoin (black circles) through the membrane is also accompanied by water migration, despite the organophilic property of the membrane used and despite the application of increasing under-pressures (white circles). Such insufficient selectivity thus does not make it possible to obtain a dehydration of acetoin at a useful level.

### Conclusion

The tests carried out demonstrate that efficient purification of acetoin is possible by selecting a hydrophilic membrane. On the other hand and counter-intuitively, the tests carried out with an organophilic membrane did not give conclusive results, in the absence of sufficient selectivity of the membrane.

1. A method for obtaining acetoin from a medium to be treated comprising acetoin, the method comprising a dehydration step by pervaporation using a hydrophilic membrane in order to obtain a treated medium comprising at least 70% acetoin, by total weight of the treated medium.

2. The method of obtaining acetoin, according to claim 1, wherein the hydrophilic membrane is selected from the group consisting of hydrophilic polymeric membranes, hydrophilic inorganic membranes, hydrophilic two-dimensional material membranes, hydrophilic mixed matrix membranes or hydrophilic membranes derived therefrom.

3. The method of obtaining acetoin, according to claim 1, in that the hydrophilic membrane is a hydrophilic silica-based membrane or a hydrophilic membrane derived therefrom.

4. The method of obtaining acetoin, according to claim 3, in that the hydrophilic silica-based membrane is obtained from organoalkoxysilane precursors.

5. The method of obtaining acetoin according to claim 1, in that an under-pressure of −700×10⁵ mPa or more is applied at the permeation surface of the hydrophilic membrane.

6. The method of obtaining acetoin, according to claim 1, in that the dehydration step is carried out in the absence of solvents and/or inorganic salts.

7. The method of obtaining acetoin, according to claim 1, comprising a step of pretreatment of the medium to be treated carried out, before the dehydration stage, using a technique chosen from the group consisting of centrifugation, frontal filtration, microfiltration and nanofiltration.

8. The method of obtaining acetoin, according to claim 1, comprising a step of post-treatment of the treated medium, which is carried out, after the dehydration step, by distillation.

9. The method of obtaining acetoin, according to claim 1, in that the medium comprising acetoin is obtained by a biological method carried out using recombinant microorganisms.

10. The method of obtaining acetoin, according to claim 9, in that the recombinant microorganisms are yeasts belonging to the genus Saccharomyces.

11. Acetoin obtained by the method according to claim 1.

12. (canceled)
